(19) United States
(12) Patent Application Publication
(10) Pub. No.: US 2026/0026788 A1
(43) Pub. Date: Jan. 29, 2026

(54) ULTRASONIC IMAGING APPARATUS AND CONTROL METHOD THEREOF

(71) Applicant: SAMSUNG MEDISON CO., LTD., Hongcheon (KR)

(72) Inventors: Jeongho HAHM, Hongcheon (KR); Junseong KIM, Hongcheon (KR); Jeheon JI, Hongcheon (KR); Changmin KIM, Hongcheon (KR)

(21) Appl. No.: 19/216,374

(22) Filed: May 22, 2025

(30) Foreign Application Priority Data
Jul. 24, 2024 (KR) 10-2024-0098136
Jan. 21, 2025 (KR) 10-2025-0008783

(51) Int. Cl. A61B 8/00; A61B 8/14

(52) U.S. Cl. CPC A61B 8/546; A61B 8/14; A61B 8/488; A61B 8/54

(57) ABSTRACT

Disclosed is an ultrasonic imaging apparatus including an electronic circuit configured to control an operation of the ultrasonic imaging apparatus, a housing configured to accommodate the electronic circuit, a cooling fan disposed inside the housing to cool the electronic circuit, and a processor configured to receive a selection of an imaging mode of the ultrasonic imaging apparatus and control a speed of the cooling fan based on the selected imaging mode.

### CROSS-REFERENCE TO RELATED APPLICATION(S)

This application is based on and claims priority under 35 U.S.C. § 119 to Korean Patent Application Nos. 10-2024-0098136 and 10-2025-0008783, filed on Jul. 24, 2024 and filed on Jan. 21, 2025 in the Korean Intellectual Property Office, the disclosure of which is incorporated by reference herein in its entirety.

### BACKGROUND

### 1. Field

The disclosure relates to an ultrasonic imaging apparatus and a control method thereof.

### 2. Description of the Related Art

Recently, in a medical field, various medical imaging apparatuses have been widely used to image and obtain information about biological tissues of a human body for the purpose of early diagnosis of various diseases or surgery. Representative examples of such medical imaging apparatuses may include ultrasonic imaging apparatuses, computed tomography (CT) apparatuses, and magnetic resonance imaging (MRI) apparatuses.

An ultrasonic imaging apparatus is a device that emits an ultrasonic signal generated from a transducer of a probe to an object, and non-invasively obtains at least one image of a region inside the object (e.g., soft tissue or blood flow) by receiving information from the signal reflected from the object. In particular, an ultrasonic imaging apparatus is used for medical purposes such as observing the inside of an object, detecting foreign substances, and measuring injury. Such an ultrasonic imaging apparatus is widely used together with other imaging diagnostic apparatuses because the ultrasonic imaging apparatus has higher stability than an imaging apparatus using an X-ray, may display images in real time, and is safe because there is no radiation exposure.

On the other hand, because an ultrasonic imaging apparatus requires high-performance signal processing and image processing, various components of the ultrasonic imaging apparatus may generate considerable heat. In order to effectively control the generated heat, the ultrasonic imaging apparatus may include a cooling fan.

Conventional speed control of a cooling fan has a reactive nature in which a speed of the cooling fan is controlled after heat has already been generated from various components. For example, conventionally, when a heating value generated from various components is greater than the maximum heating value generated, the speed of a cooling fan is increased.

However, the speed control of such a conventional cooling fan does not reflect an actual usage environment of an ultrasonic imaging apparatus, which causes unnecessary noise.

### SUMMARY

It is an aspect of the disclosure to provide an ultrasonic imaging apparatus capable of controlling a speed of a cooling fan depending on an imaging mode and a control method thereof.

Additional aspects of the disclosure will be set forth in part in the description which follows and, in part, will be obvious from the description, or may be learned by practice of the disclosure.

An aspect of the disclosure provides an ultrasonic imaging apparatus including an electronic circuit configured to control an operation of the ultrasonic imaging apparatus, a housing configured to accommodate the electronic circuit, a cooling fan disposed inside the housing to cool the electronic circuit, and a processor configured to receive a selection of an imaging mode of the ultrasonic imaging apparatus and control a speed of the cooling fan based on the selected imaging mode.

The processor may be configured to change the speed of the cooling fan from a first speed to a second speed in response to the selected imaging mode being switched from a first imaging mode to a second imaging mode.

The processor may be configured to control the speed of the cooling fan based on a pre-stored lookup table and the selected imaging mode.

The pre-stored lookup table may be a table in which a target rotation speed for each of a plurality of imaging modes is mapped in proportion to a heating value generated from the electronic circuit while operating in the plurality of imaging modes.

The processor may be configured to reduce the speed of the cooling fan based on receiving a Freeze signal while operating in the selected imaging mode.

The processor may be configured to receive a selection of a diagnostic region of an object and control the speed of the cooling fan based on the selected imaging mode and the selected diagnostic region of the object.

The processor may be configured to control the speed of the cooling fan to a first speed based on the selected imaging mode being a first imaging mode and the selected diagnostic region of the object being a first region, and control the speed of the cooling fan to a second speed based on the selected imaging mode being the first imaging mode and the selected diagnostic region of the object being a second region different from the first region.

The processor may be configured to control the speed of the cooling fan to a first speed based on the selected imaging mode being a B-mode and the selected diagnostic region of the object being a first region, control the speed of the cooling fan to a second speed based on the selected imaging mode being the B-mode and the selected diagnostic region of the object being a second region, and control the speed of the cooling fan to a third speed based on the selected imaging mode being a Doppler mode and the selected diagnostic region of the object being the first region or the second region.

The third speed may be faster than the first speed and slower than the second speed.

The processor may be configured to control the speed of the cooling fan to a target speed corresponding to a Doppler mode regardless of the selected diagnostic region when the selected imaging mode is the Doppler mode.

The processor may be configured to control the speed of the cooling fan to a preset lowest speed during a booting operation of booting a program necessary to execute the imaging mode.

The processor may be configured to maintain the speed of the cooling fan at the preset lowest speed for a predetermined period of time in response to the ultrasonic imaging apparatus being powered off.

Another aspect of the disclosure provides a control method of an ultrasonic imaging apparatus which includes an electronic circuit configured to control an operation of the ultrasonic imaging apparatus, a housing configured to accommodate the electronic circuit, and a cooling fan disposed inside the housing to cool the electronic circuit, wherein the control method includes receiving a selection of an imaging mode of the ultrasonic imaging apparatus and controlling a speed of the cooling fan based on the selected imaging mode.

The control method may further include changing the speed of the cooling fan from a first speed to a second speed in response to the selected imaging mode being switched from a first imaging mode to a second imaging mode.

The controlling of the speed of the cooling fan based on the selected imaging mode may include controlling the speed of the cooling fan based on a pre-stored lookup table and the selected imaging mode.

The pre-stored lookup table may be a table in which a target rotation speed for each of a plurality of imaging modes is mapped in proportion to a heating value generated from the electronic circuit while operating in the plurality of imaging modes.

The control method may further include reducing the speed of the cooling fan based on receiving a Freeze signal while operating in the selected imaging mode.

The controlling of the speed of the cooling fan based on the selected imaging mode may include receiving a selection of a diagnostic region of an object, and controlling the speed of the cooling fan based on the selected imaging mode and the selected diagnostic region of the object.

The controlling of the speed of the cooling fan based on the selected imaging mode and the selected diagnostic region of the object may include controlling the speed of the cooling fan to a first speed based on the selected imaging mode being a first imaging mode and the selected diagnostic region of the object being a first region, and controlling the speed of the cooling fan to a second speed based on the selected imaging mode being the first imaging mode and the selected diagnostic region of the object being a second region different from the first region.

The controlling of the speed of the cooling fan based on the selected imaging mode and the selected diagnostic region of the object may include controlling the speed of the cooling fan to a first speed based on the selected imaging mode being a B-mode and the selected diagnostic region of the object being a first region, controlling the speed of the cooling fan to a second speed based on the selected imaging mode being the B-mode and the selected diagnostic region of the object being a second region, and controlling the speed of the cooling fan to a third speed based on the selected imaging mode being a Doppler mode and the selected diagnostic region of the object being the first region or the second region.

The third speed may be faster than the first speed and slower than the second speed.

The controlling of the speed of the cooling fan based on the selected imaging mode and the selected diagnostic region of the object may include controlling the speed of the cooling fan to a target speed corresponding to the Doppler mode regardless of the selected diagnostic region when the selected imaging mode is the Doppler mode.

The control method may further include controlling the speed of the cooling fan to a preset lowest speed during a booting operation of booting a program necessary to execute the imaging mode.

The control method may further include maintaining the speed of the cooling fan at the preset lowest speed for a predetermined period of time in response to the ultrasonic imaging apparatus being powered off.

### BRIEF DESCRIPTION OF THE DRAWINGS

These and/or other aspects of the disclosure will become apparent and more readily appreciated from the following description of the embodiments, taken in conjunction with the accompanying drawings of which:

FIGS. 1 and 2 are block diagrams illustrating components of an ultrasonic imaging system according to an embodiment;

FIGS. 3 to 6 are views illustrating the ultrasonic imaging system according to an embodiment;

FIG. 7 is a block diagram illustrating components of an ultrasonic imaging apparatus according to an embodiment;

FIG. 8 is a flowchart illustrating an example of a control method of the ultrasonic imaging apparatus according to an embodiment;

FIG. 9 is a graph for explaining an example of controlling a speed of a cooling fan according to an embodiment;

FIG. 10 is a graph for explaining a heating value generated depending on an imaging mode;

FIG. 11 is a graph for explaining the heating value generated depending on a diagnostic region of an object in a selected imaging mode;

FIG. 12 illustrates a lookup table used for speed control of the cooling fan; and

FIG. 13 is a flowchart illustrating an example of a control method of the ultrasonic imaging apparatus according to an embodiment.

### DETAILED DESCRIPTION

This disclosure will explain the principles and disclose embodiments of the disclosure to clarify the scope of the claims of the disclosure and enable those skilled in the art to which the embodiments of the disclosure belong to practice the embodiments. The embodiments of the disclosure may be implemented in various forms.

Throughout the specification, like reference numbers refer to like elements throughout this specification. This specification does not describe all components of the embodiments, and general contents in the technical field to which the disclosure belongs or overlapping contents between the embodiments will not be described. The “module” or “unit” used in the specification may be implemented as one or a combination of two or more of software, hardware, or firmware, and according to embodiments, a plurality of “module” or “unit” may be implemented as a single element, or a single “module” or “unit” may include a plurality of elements.

The singular form of a noun corresponding to an item may include a single item or a plurality of items, unless the relevant context clearly indicates otherwise.

In this disclosure, each of phrases such as “A or B,” “at least one of A and B,” “at least one of A or B,” “A, B or C,” “at least one of A, B and C,” and “at least one of A, B, or C” may include any one of the items listed together in the corresponding one of the phrases, or all possible combinations thereof.

The term “and/or” includes any combination of a plurality of related components or any one of a plurality of related components.

The terms such as “first,” “second,” “primary,” and “secondary” may simply be used to distinguish a given component from other corresponding components, and do not limit the corresponding components in any other respect (e.g., importance or order).

The terms “front surface,” “rear surface,” “upper surface,” “lower surface,” “side surface,” “left side,” “right side,” “upper portion,” “lower portion,” and the like used in the disclosure are defined with reference to the drawings, and the shape and position of each component are not limited by these terms.

The terms “comprises,” “has,” and the like are intended to indicate that there are features, numbers, steps, operations, components, parts, or combinations thereof described in the disclosure, and do not exclude the presence or addition of one or more other features, numbers, steps, operations, components, parts, or combinations thereof.

When any component is referred to as being “connected,” “coupled,” “supported,” or “in contact” with another component, this includes a case in which the components are indirectly connected, coupled, supported, or in contact with each other through a third component as well as directly connected, coupled, supported, or in contact with each other.

When any component is referred to as being located “on” or “over” another component, this includes not only a case in which any component is in contact with another component but also a case in which another component is present between the two components.

Hereinafter, an ultrasonic apparatus according to various embodiments will be described in detail with reference to the accompanying drawings. When described with reference to the accompanying drawings, similar reference numbers may be assigned to identical or corresponding components and redundant description thereof may be omitted.

In this disclosure, an image may include a medical image acquired by a medical imaging apparatus such as a magnetic resonance imaging (MRI) apparatus, a computed tomography (CT) apparatus, an ultrasonic imaging apparatus, and an X-ray imaging apparatus.

In this disclosure, an ‘object’, which is subject to photography, may include a person, animal, or part thereof. For example, the object may include a part of a human body (an organ, etc.) or a phantom.

In this disclosure, an ‘ultrasonic image’ refers to an image of an object that has been generated or processed based on an ultrasonic signal transmitted to and reflected from the object.

Hereinafter, embodiments will be described in detail with reference to the drawings.

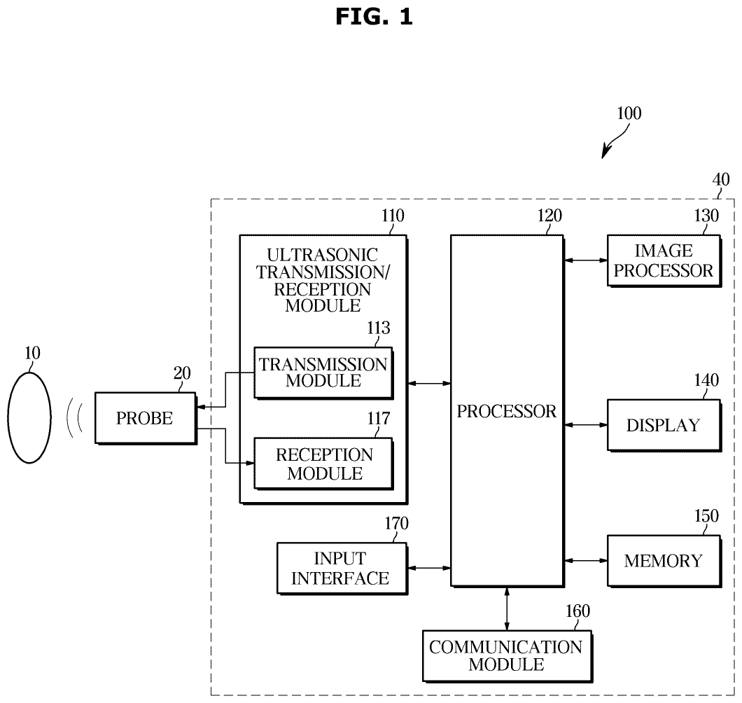

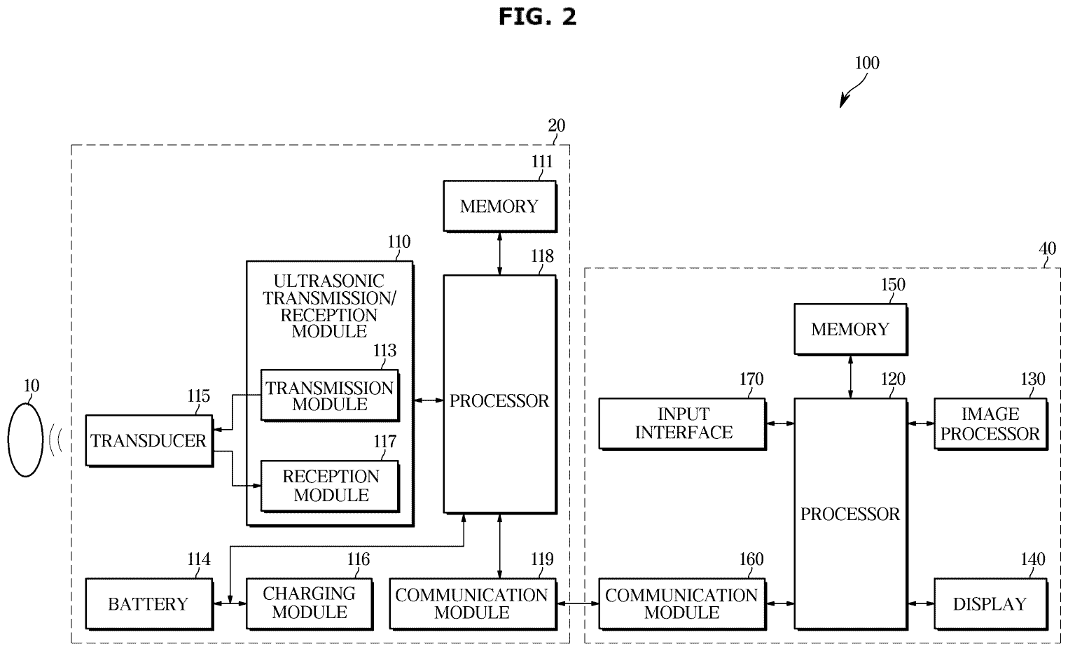

FIGS. 1 and 2 are block diagrams illustrating components of an ultrasonic imaging system according to an embodiment.

Referring to FIGS. 1 and 2, an ultrasonic imaging system 100 may include a probe 20 and an ultrasonic imaging apparatus 40.

The ultrasonic imaging apparatus 40 may be implemented not only in a cart type but also in a portable type. A portable ultrasonic imaging apparatus may include, for example, a smart phone, a laptop computer, a personal digital assistant (PDA), a tablet PC, etc., which include a probe and an application, but is not limited thereto.

The probe 20 may include a wired probe connected to the ultrasonic imaging apparatus 40 by wire to communicate with the ultrasonic imaging apparatus 40 by wire, a wireless probe wirelessly connected to the ultrasonic imaging apparatus 40 to communicate wirelessly with the ultrasonic imaging apparatus 40, and/or a hybrid probe connected to the ultrasonic imaging apparatus 40 by wire or wirelessly to communicate with the ultrasonic imaging apparatus 40 by wire or wirelessly.

According to various embodiments, as illustrated in FIG. 1, the ultrasonic imaging apparatus 40 may include an ultrasonic transmission/reception module 110, and as illustrated in FIG. 2, the probe 20 may include the ultrasonic transmission/reception module 110. According to various embodiments, both the ultrasonic imaging apparatus 40 and the probe 20 may also include the ultrasonic transmission/reception module 110.

According to various embodiments, the probe 20 may further include an image processor 130, a display 140, and/or an input interface 170.

Accordingly, descriptions of the ultrasonic transmission/reception module 110, the image processor 130, the display 140, and/or the input interface 170 included in the ultrasonic imaging apparatus 40 may also be applied to the ultrasonic transmission/reception module 110, the image processor 130, the display 140, and/or the input interface 170 included in the probe 20.

FIG. 1 is a block diagram illustrating components of the ultrasonic imaging system 100 in a case in which the probe 20 is a wired probe or a hybrid probe.

The probe 20 may include a plurality of transducers. The plurality of transducers may transmit an ultrasonic signal to an object 10 in response to a transmission signal applied from a transmission module 113. The plurality of transducers may form a reception signal by receiving the ultrasonic signal (echo signal) reflected from the object 10. The probe 20 may be implemented as an integrated type with the ultrasonic imaging apparatus 40, or may be implemented as a separate type connected to the ultrasonic imaging apparatus 40 by wire. The ultrasonic imaging apparatus 40 may be connected to the one or more probes 20 depending on the implementation type.

In the case in which the probe 20 is a wired probe or a hybrid probe, the probe 20 may include a cable and a connector connectable to a connector of the ultrasonic imaging apparatus 40.

The probe 20 according to an embodiment may be implemented as a two-dimensional probe. In a case in which the probe 20 is implemented as a two-dimensional probe, the plurality of transducers included in the probe 20 may be arranged in two dimensions to form a two-dimensional transducer array.

For example, the two-dimensional transducer array may have a form in which a plurality of sub-arrays including the plurality of transducers arranged in a first direction is arranged in a second direction different from the first direction.

Also, in the case in which the probe 20 according to an embodiment is implemented as a two-dimensional probe, the ultrasonic transmission/reception module 110 may include an analog beamformer and a digital beamformer, Or, the two-dimensional probe may include one or both of the analog beamformer and the digital beamformer depending on the implementation type.

A processor 120 controls the transmission module 113 to form a transmission signal to be applied to each of transducers 115 in consideration of positions and focused points of the plurality of transducers included in the probe 20.

The processor 120 may control a reception module 117 to generate ultrasonic data by converting reception signals received from the probe 20 into analog to digital and summing up the digitally converted reception signals in consideration of the positions and focused points of the plurality of transducers.

In the case in which the probe 20 is implemented as a two-dimensional probe, the processor 120 may calculate a time delay value for digital beamforming by each sub-array for each of the plurality of sub-arrays included in the two-dimensional transducer array. The processor 120 may also calculate a time delay value for analog beamforming for each of the transducers included in one of the plurality of sub-arrays. The processor 120 may control the analog beamformer and the digital beamformer to form a transmission signal to be applied to each of the plurality of transducers depending on the time delay values for analog beamforming and the time delay values for digital beamforming. The processor 120 may also control the analog beamformer to sum up the signals received from the plurality of transducers by each sub-array depending on the time delay values for analog beamforming. The processor 120 may also control the ultrasonic transmission/reception module 110 to convert the summed signal by each sub-array into analog to digital. The processor 120 may also control the digital beamformer to generate ultrasonic data by summing up the digitally converted signals depending on the time delay values for digital beamforming.

The image processor 130 may generate an ultrasonic image using the generated ultrasonic data.

The display 140 may display the generated ultrasonic image and a variety of information processed in the ultrasonic imaging apparatus 40 and/or the probe 20. The probe 20 and/or the ultrasonic imaging apparatus 40 may include the one or more displays 140 depending on the implementation type. The display 140 may also include a touch panel or a touch screen.

The processor 120 may control the overall operation of the ultrasonic imaging apparatus 40 and signal flows between internal components of the ultrasonic imaging apparatus 40. The processor 120 may perform or control various operations or functions of the ultrasonic imaging apparatus 40 by executing programs or instructions stored in memory 150. The processor 120 may also control an operation of the ultrasonic imaging apparatus 40 by receiving a control signal from the input interface 170 or an external device.

The ultrasonic imaging apparatus 40 may include a communication module 160, and may be connected with an external device (e.g., the probe 20, a server, a medical device, a portable device (a smart phone, tablet PC, wearable device, etc.)) through the communication module 160.

The communication module 160 may include one or more components enabling communication with the external device, and may include, for example, at least one of a short-range communication module, a wired communication module, and a wireless communication module.

The communication module 160 may also receive a control signal and data from an external device and transmit the received control signal to the processor 120 so that the processor 120 may control the ultrasonic imaging apparatus 40 in response to the received control signal.

Alternatively, the processor 120 may transmit a control signal to an external device through the communication module 160, so that the external device may be controlled according to the control signal of the processor 120.

For example, the external device may process data in the external device according to the control signal of the processor 120 received through the communication module.

A program capable of controlling the ultrasonic imaging apparatus 40 may be installed in the external device, and the program may include instructions for performing part or all of the operations of the processor 120.

The program may be pre-loaded in the external device, or a user of the external device may download and install the program from a server providing an application. The server providing the application may include a storage medium in which the program is stored.

The memory 150 may store various data or programs for driving and controlling the ultrasonic imaging apparatus 40, inputted and outputted ultrasonic data, ultrasonic images, and the like.

The input interface 170 may receive user input for controlling the ultrasonic imaging apparatus 40. For example, the user input may include, but is not limited to, input of manipulating a button, a keypad, a mouse, a trackball, a jog switch, a knob, and the like, input of touching a touch pad or touch screen, voice input, motion input, biometric information input (e.g., iris recognition, fingerprint recognition, etc.), and the like.

FIG. 2 illustrates a control block diagram of the ultrasonic imaging system 100 in a case in which the probe 20 is a wireless probe or a hybrid probe.

According to various embodiments, the ultrasonic imaging apparatus 40 illustrated in FIG. 2 may be replaced with the ultrasonic imaging apparatus 40 described with reference to FIG. 1.

According to various embodiments, the probe 20 described with reference to FIG. 1 may be replaced with the probe 20 to be described with reference to FIG. 2.

The probe 20 may include the transmission module 113, a battery 114, the transducer 115, a charging module 116, the reception module 117, a processor 118, and a communication module 119. FIG. 2 illustrates that the probe 20 includes both the transmission module 113 and the reception module 117, but the probe 20 may include only part of configurations of the transmission module 113 and the reception module 117 depending on the implementation type, and the part of the configurations of the transmission module 113 and the reception module 117 may be included in the ultrasonic imaging apparatus 40. Additionally, the probe 20 may further include the image processor 130.

The transducer 115 may include a plurality of transducers. The plurality of transducers may transmit ultrasonic signals to the object 10 in response to transmission signals applied from the transmission module 113. The plurality of transducers may also receive ultrasonic signals reflected from the object 10 to form reception signals.

The charging module 116 may charge the battery 114. The charging module 116 may receive electric power from the outside. The charging module 116 may receive electric power wirelessly. However, the charging module 116 is not limited thereto, and may receive electric power by wire. The charging module 116 may transfer the received electric power to the battery 114.

The processor 118 may control the transmission module 113 to form a transmission signal to be applied to each of the plurality of transducers in consideration of the positions and focused points of the plurality of transducers.

The processor 118 controls the reception module 117 to generate ultrasonic data by converting reception signals received from the transducer 115 into analog to digital and summing up the digitally converted reception signals in consideration of the positions and focused points of the plurality of transducers. Alternatively, in a case in which the probe 20 includes the image processor 130, the probe 20 may generate an ultrasonic image using the generated ultrasonic data.

In the case in which the probe 20 is implemented as a two-dimensional probe, the processor 118 may calculate the time delay value for digital beamforming by each sub-array for each of the plurality of sub-arrays included in the two-dimensional transducer array. The processor 118 may also calculate the time delay value for analog beamforming for each of the transducers included in one of the plurality of sub-arrays. The processor 118 may control the analog beamformer and the digital beamformer to form a transmission signal to be applied to each of the plurality of transducers depending on the time delay values for analog beamforming and the time delay values for digital beamforming. The processor 118 may also control the analog beamformer to sum up the signals received from the plurality of transducers by each sub-array depending on the time delay values for analog beamforming. The processor 118 may also control the ultrasonic transmission/reception module 110 to convert the summed signal by each sub-array into analog to digital. The processor 118 may also control the digital beamformer to generate ultrasonic data by summing up the digitally converted signals depending on the time delay values for digital beamforming.

The processor 118 may control the overall operation of the probe 20 and signal flows between internal components of the probe 20. The processor 118 may perform or control various operations or functions of the probe 20 by executing programs or instructions stored in memory 111. The processor 118 may also control an operation of the probe 20 by receiving a control signal from the input interface 170 of the probe 20 or an external device (e.g., the ultrasonic imaging apparatus 40).

The communication module 119 may wirelessly transmit the generated ultrasonic data or ultrasonic images to the ultrasonic imaging apparatus 40 through a wireless network. The communication module 119 may also receive a control signal and data from the ultrasonic imaging apparatus 40.

The ultrasonic imaging apparatus 40 may receive the ultrasonic data and/or ultrasonic images from the probe 20.

In an embodiment, the case in which the probe 20 includes the image processor 130 capable of generating ultrasonic images using the ultrasonic data, the probe 20 may transmit the ultrasonic data and/or the ultrasonic images generated by the image processor 130 to the ultrasonic imaging apparatus 40.

In an embodiment, a case in which the probe 20 does not include the image processor 130 capable of generating ultrasonic images using the ultrasonic data, the probe 20 may transmit the ultrasonic data to the ultrasonic imaging apparatus 40. The ultrasonic data may include ultrasonic raw data, and the ultrasonic images may refer to ultrasonic image data.

The ultrasonic imaging apparatus 40 may include the processor 120, the image processor 130, the display 140, the memory 150, the communication module 160, and the input interface 170.

The image processor 130 generates ultrasonic images using the ultrasonic data received from the probe 20.

The display 140 may display the ultrasonic images received from the probe 20, ultrasonic images generated by processing the ultrasonic data received from the probe 20, and a variety of information processed by the ultrasonic imaging system 100. The ultrasonic imaging apparatus 40 may include the one or more displays 140 depending on the implementation type. The display 140 may also include a touch panel or a touch screen.

The processor 120 may control the overall operation of the ultrasonic imaging apparatus 40 and signal flows between the internal components of the ultrasonic imaging apparatus 40. The processor 120 may perform or control the various operations or functions of the ultrasonic imaging apparatus 40 by executing the programs or applications stored in the memory 150. The processor 120 may also control the operation of the ultrasonic imaging apparatus 40 by receiving a control signal from the input interface 170 or an external device.

The ultrasonic imaging apparatus 40 may include the communication module 160, and may be connected with an external device (e.g., the probe 20, a server, a medical device, a portable device (a smart phone, tablet PC, wearable device, etc.)) through the communication module 160.

The communication module 160 may include one or more components that enable communication with the external device, and may include, for example, at least one of a short-range communication module, a wired communication module, and a wireless communication module.

The communication module 160 of the ultrasonic imaging apparatus 40 and the communication module 119 of the probe 20 may communicate using a network or a short-range wireless communication method. For example, the communication module 160 of the ultrasonic imaging apparatus 40 and the communication module 119 of the probe 20 may communicate using any one of wireless LAN, Wi-Fi, Bluetooth, ZigBee, Wi-Fi Direct (WFD), Infrared Data Association (IrDA), Bluetooth Low Energy (BLE), Near Field Communication (NFC), Wireless Broadband Internet (WiBro), World Interoperability for Microwave Access (WiMAX), Shared Wireless Access Protocol (SWAP), Wireless Gigabit Alliance (WiGig), RF communication, and a wireless data communication method including 60 GHz millimeter wave (mm wave) short-range communication.

To this end, the communication module 160 of the ultrasonic imaging apparatus 40 and the communication module 119 of the probe 20 may include at least one of a wireless LAN communication module, a Wi-Fi communication module, a Bluetooth communication module, a ZigBee communication module, a Wi-Fi Direct (WFD) communication module, an Infrared Data Association (IrDA) communication module, a Bluetooth Low Energy (BLE) communication module, a Near Field Communication (NFC) module, a Wireless Broadband Internet (WiBro) communication module, a World Interoperability for Microwave Access (WiMAX) communication module, a Shared Wireless Access Protocol (SWAP) communication module, a Wireless Gigabit Alliance (WiGig) communication module, a RF communication module, and a 60 GHz millimeter wave (mm wave) short-range communication module.

In an embodiment, the probe 20 may transmit device information (e.g., ID information) of the probe 20 using a first communication method (e.g., BLE), may be wirelessly paired with the ultrasonic imaging apparatus 40, and may transmit ultrasonic data and/or ultrasonic images to the paired ultrasonic imaging apparatus 40.

The device information of the probe 20 may include a variety of information related to a serial number, model name, battery state of the probe 20, and the like.

The ultrasonic imaging apparatus 40 may receive the device information (e.g., ID information) of the probe 20 from the probe 20 using the first communication method (e.g., BLE), may be wirelessly paired with the probe 20, may transmit an activation signal to the paired probe 20, and may receive the ultrasonic data and/or ultrasonic images from the probe 20. In this case, the activation signal may include a signal for controlling the operation of the probe 20.

In an embodiment, the probe 20 may transmit the device information (e.g., ID information) of the probe 20 using the first communication method (e.g., BLE), may be wirelessly paired with the ultrasonic imaging apparatus 40, and may transmit the ultrasonic data and/or ultrasonic images to the ultrasonic imaging apparatus 40 paired by the first communication method using a second communication method (e.g., 60 GHz millimeter wave and Wi-Fi).

The ultrasonic imaging apparatus 40 may receive the device information (e.g., ID information) of the probe 20 from the probe 20 using the first communication method (e.g., BLE), may be wirelessly paired with the probe 20, may transmit the activation signal to the paired probe 20, and receive the ultrasonic data and/or ultrasonic images from the probe 20 using the second communication method (e.g., 60 GHz millimeter wave and Wi-Fi).

According to various embodiments, the first communication method used to pair the probe 20 and the ultrasonic imaging apparatus 40 with each other may have a frequency band lower than a frequency band of the second communication method used by the probe 20 to transmit the ultrasonic data and/or ultrasonic images to the ultrasonic imaging apparatus 40.

The display 140 of the ultrasonic imaging apparatus 40 may display UIs (user interfaces) indicating the device information of the probe 20. For example, the display 140 may display UIs, which indicate identification information of the wireless ultrasonic probe 20, a pairing method indicating a pairing method with the probe 20, a data communication state between the probe 20 and the ultrasonic imaging apparatus 40, a method of performing data communication with the ultrasonic imaging apparatus 40, and the battery state of the probe 20.

In a case in which the probe 20 includes the display 140, the display 140 of the probe 20 may display the UIs indicating the device information of the probe 20. For example, the display 140 may display the UIs, which indicate the identification information of the wireless ultrasonic probe 20, the pairing method indicating the pairing method with the probe 20, the data communication state between the probe 20 and the ultrasonic imaging apparatus 40, the method of performing the data communication with the ultrasonic imaging apparatus 40, and the battery state of the probe 20.

The communication module 160 may also receive a control signal and data from an external device and transmit the received control signal to the processor 120 so that the processor 120 may control the ultrasonic imaging apparatus 40 in response to the received control signal.

Alternatively, the processor 120 may transmit a control signal to an external device through the communication module 160, so that the external device may be controlled according to the control signal of the processor 120.

For example, the external device may process data in the external device according to the control signal of the processor 120 received through the communication module.

The program capable of controlling the ultrasonic imaging apparatus 40 may be installed in the external device, and the program may include instructions for performing part or all of the operations of the processor 120.

The program may be pre-loaded in the external device, or the user of the external device may download and install the program from the server providing the application. The server providing the application may include the storage medium in which the program is stored.

The memory 150 may store various data or programs for driving and controlling the ultrasonic imaging apparatus 40, inputted and outputted ultrasonic data, ultrasonic images, and the like.

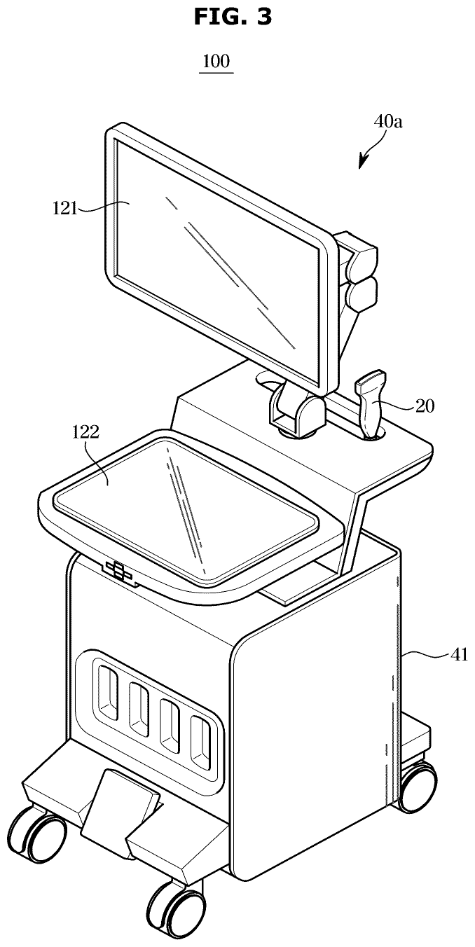

Examples of the ultrasonic imaging system 100 according to an embodiment of the disclosure will be described later with reference to FIGS. 3 to 6.

FIGS. 3 to 6 are views illustrating the ultrasonic imaging system according to an embodiment.

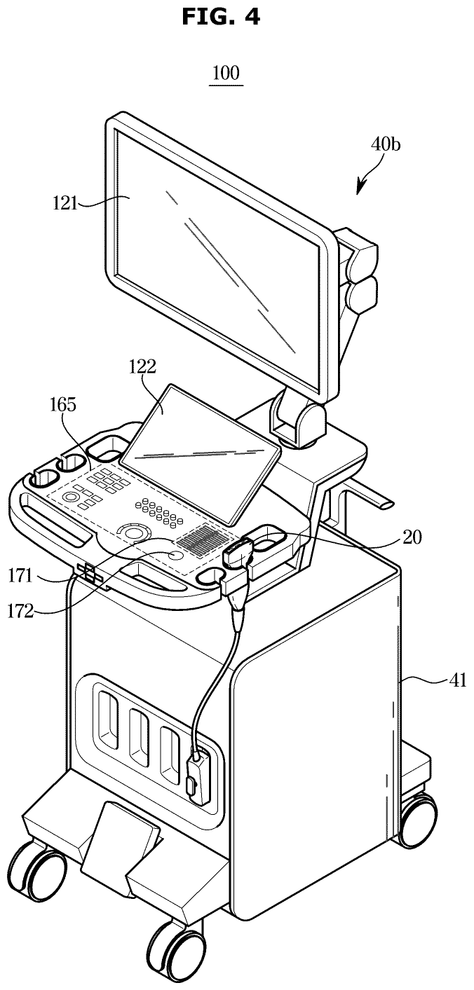

Referring to FIGS. 3 and 4, ultrasonic imaging apparatuses 40a and 40b may include a main display 121 (140) and a sub display 122 (140). At least one of the main display 121 and the sub display 122 may be implemented as a touch screen. Also, at least one of the main display 121 and the sub display 122 may be implemented as a touch screen, and may receive input of data for controlling the ultrasonic imaging apparatuses 40a and 40b from a user by providing GUIs. For example, the main display 121 may display ultrasonic images, and the sub display 122 may display a control panel for controlling the display of the ultrasonic images in the form of GUIs. The sub display 122 may receive input of data for controlling the display of images through the control panel displayed in the form of GUIs. For example, a time gain compensation (TGC) button, a Freeze button, a trackball, a jog switch, a knob, and the like may be provided as GUIs on the sub display 122.

The ultrasonic imaging apparatuses 40a and 40b may control the display of ultrasonic images displayed on the main display 121 using the inputted control data. The ultrasonic imaging apparatuses 40a and 40b may be connected to the probe 20 by wire or wirelessly to transmit and receive ultrasonic signals to and from the object 10.

Referring to FIG. 4, the ultrasonic imaging apparatus 40b may further include a control panel 165 in addition to the main display 121 and the sub display 122. The control panel 165 may include a button, a trackball, a jog switch, a knob, and the like, and may receive input of data for controlling the ultrasonic imaging apparatus 40b from the user. For example, the control panel 165 may include a TGC button 171, a Freeze button 172, and the like. The TGC button 171 is a button for setting a TGC value by each of depths of ultrasonic images. The ultrasonic imaging apparatus 40b may keep a state in which a frame image at that point in time is displayed when the Freeze button 172 input is detected while scanning an ultrasonic image.

The trackball, jog switch, knob, and the like included in the control panel 165 may be provided as GUIs on the main display 121 or the sub display 122. The ultrasonic imaging apparatuses 40a and 40b may be connected to the probe 20 to transmit and receive ultrasonic signals to and from the object 10.

Referring to FIGS. 3 and 4, the ultrasonic imaging apparatuses 40a and 40b may include a housing 41.

The housing 41 may accommodate various components of the ultrasonic imaging apparatuses 40a and 40b. The housing 41 may be provided in a box shape at a lower end of the ultrasonic imaging apparatuses 40a and 40b. The housing 41 may be designed with a structure for protection and heat management of various components accommodated therein. For example, vent holes may be formed on the housing 41 to circulate air between the outside of the housing 41 and the inside of the housing 41. As another example, a material of the housing 41 may be a material allowing heat to be effectively transferred from the inside of the housing 41 to the outside of the housing 41.

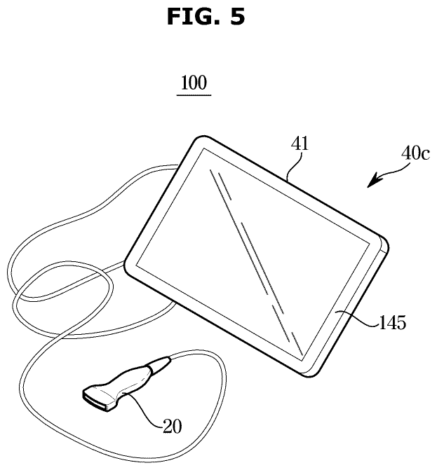

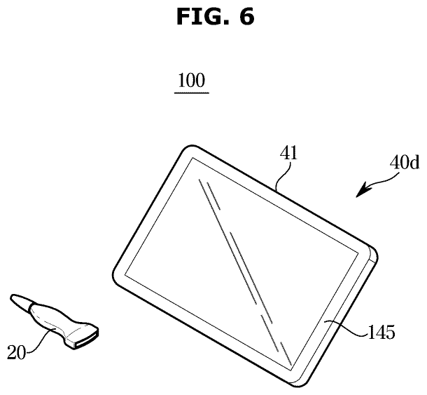

Referring to FIGS. 5 and 6, an ultrasonic imaging apparatus 40c may be implemented in a portable type. The portable ultrasonic imaging apparatus 40c may include, for example, smart phones, laptop computers, PDAs, or tablet PCs, which include probes and applications, but is not limited thereto.

The ultrasonic imaging apparatus 40c may include the housing 41. Referring to FIG. 5, the probe 20 may be connected to one side of the housing 41 by wire. To this end, the housing 41 may include a connection terminal to and from which a cable connected to the probe 20 may be attached and detached, and the probe 20 may include a connection terminal to and from which a cable connected to the connection terminal provided on the housing 41 may be attached and detached.

Referring to FIG. 6, the probe 20 may be wirelessly connected to the ultrasonic imaging apparatus 40d. An input/output interface (e.g., a touch screen) 145 (140 and 170) may be provided on the housing 41. Ultrasonic images, a variety of information processed in the ultrasonic imaging apparatus, and GUIs may be displayed on the input/output interface 145.

Also, an ultrasonic image may be displayed on the input/output interface 145. The ultrasonic imaging apparatus 40c may correct the ultrasonic image displayed on the input/output interface 145 using AI. The ultrasonic imaging apparatus 40c may provide an alarm notifying information about a lesion in the ultrasonic image displayed on the input/output interface 145 through various audiovisual tools such as graphics, sounds, and vibrations by using the AI.

The ultrasonic imaging apparatus 40c may output a control panel displayed in the form of a GUI through the input/output interface 145.

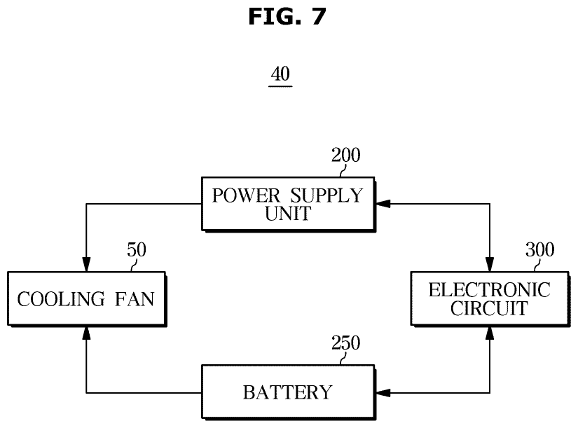

FIG. 7 is a block diagram illustrating components of an ultrasonic imaging apparatus according to an embodiment.

Referring to FIG. 7, the ultrasonic imaging apparatus 40 according to an embodiment may include an electronic circuit 300 configured to control the operation of the ultrasonic imaging apparatus 40, a cooling fan 50 configured to cool the electronic circuit 300, a power supply unit 200 configured to supply power to the electronic circuit 300 and the cooling fan 50, and/or a battery 250 configured to supply power to the electronic circuit 300 and the cooling fan 50 when the ultrasonic imaging apparatus 40 is powered off.

The electronic circuit 300 may include electrical circuits formed by various components of the ultrasonic imaging apparatus 40. For example, the electronic circuit 300 may include circuit configurations for implementing the ultrasonic transmission/reception module 110, the processor 120, the communication module 160, the image processor 130, the display 140, and the memory 150, which are illustrated in FIG. 1.

Specifically, the electronic circuit 300 may be a circuit configuration including a CPU, a Micom, a memory device, and a communication module device, respectively.

In an embodiment, the electronic circuit 300 may be accommodated in the housing 41. For example, the electronic circuit 300 may be disposed on an upper side of the housing 41.

However, the arrangement of the electronic circuit 300 is not limited thereto, and may be disposed inside the housing 41 according to various embodiments. For example, the electronic circuit 300 may be disposed on a lower side inside the housing 41.

The electronic circuit 300 may generate heat in a process of generating a control signal for controlling the operation of the ultrasonic imaging apparatus 40. Because such heat generation may cause performance degradation or malfunction of the electronic circuit 300, it is necessary to effectively cool the heat generated from the electronic circuit 300.

In an embodiment, the cooling fan 50 may be a component for cooling the electronic circuit 300. For example, the cooling fan 50 may cool the electronic circuit 300 by discharging air heated by heat generated from the electronic circuit 300 to the outside through the vent holes formed on the housing 41 and introducing and circulating cool air from outside into the housing 41.

The cooling fan 50 may include a cooling fan motor. The cooling fan motor may include a brushless DC (BLDC) motor, an AC induction motor, a DC servo motor, or a stepper motor.

The cooling fan 50 may be disposed inside the housing 41. The cooling fan 50 may be disposed at various locations inside the housing 41. For example, the cooling fan 50 may be disposed on an upper or lower side inside the housing 41.

A plurality of the cooling fans 50 may be configured. For example, the plurality of cooling fans 50 may be disposed inside the housing 41.

In a case in which the plurality of cooling fans 50 is configured, the processor 120 may control a speed of each of the plurality of cooling fans 50. For example, the processor 120 may increase the speeds of the plurality of cooling fans 50 at the same speed and may decrease the speed of the plurality of cooling fans 50 at the same speed. Also, the processor 120 may increase the speeds of only some of the plurality of cooling fans 50, and may decrease the speeds of only some of the plurality of cooling fans 50.

The power supply unit 200 may supply power to the electronic circuit 300 and the cooling fan 50 when the ultrasonic imaging apparatus 40 is powered on. For example, the power supply unit 200 may supply power to the electronic circuit 300 and the cooling fan 50 under the control of the processor 120 based on the ultrasonic imaging apparatus 40 being powered on.

The battery 250 may supply power to the electronic circuit 300 and the cooling fan 50 when the ultrasonic imaging apparatus 40 is powered off.

For example, the battery 250 may supply power to the electronic circuit 300 and the cooling fan 50 to perform an afterimage removal function of the display 140 under the control of the processor 120 based on the ultrasonic imaging apparatus 40 being powered off.

The ultrasonic imaging apparatus 40 may perform an afterimage removal operation of the display 140 in various ways. For example, after the ultrasonic imaging apparatus 40 is powered off, the ultrasonic imaging apparatus 40 may remove afterimages in the display 140 by sequentially displaying RGB patterns on all pixels of the display 140 or by displaying the entire screen in white and then black.

As another example, the ultrasonic imaging apparatus 40 may remove afterimages on the display 140 by causing all the pixels on the display 140 to emit right at maximum brightness for a certain period of time or by repeatedly displaying an image of a specific pattern.

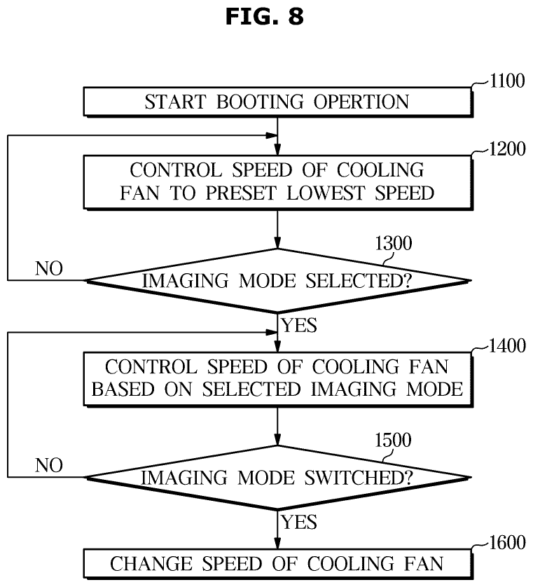

FIG. 8 is a flowchart illustrating an example of a control method of the ultrasonic imaging apparatus according to an embodiment,

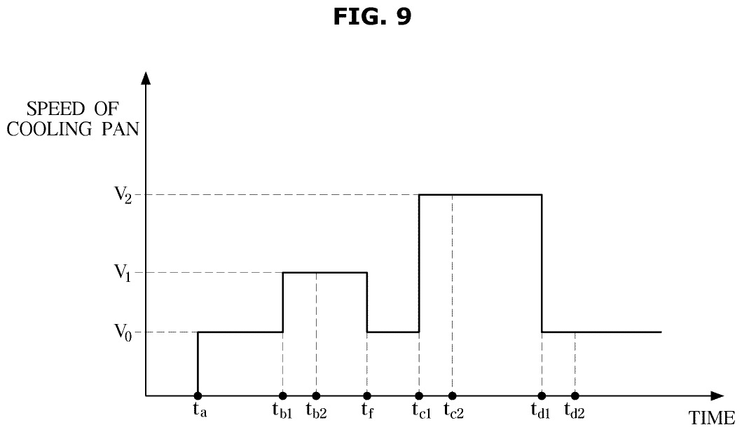

FIG. 9 is a graph for explaining an example of controlling a speed of a cooling fan according to an embodiment.

Referring to FIG. 8, in an embodiment, the processor 120 may start a booting operation based on power being turned on (1100).

The booting operation may include an operation for executing programs necessary to execute an imaging mode. For example, the booting operation may include an operation of executing a basic input/output system (BIOS), an operation of loading an operating system, and/or an operation of loading a program for executing the imaging mode.

The operation of executing the BIOS may include an operation of performing a Power-On Self-Test (POST) to check states of various hardware of the ultrasonic imaging apparatus 40 and performing a preparation process for loading the operating system.

The operation of loading the operating system may include loading the operating system that manages the operation of the ultrasonic imaging apparatus 40.

The program for executing the imaging mode may include an ultrasonic imaging program for obtaining an ultrasonic image according to the imaging mode, storing the obtained ultrasonic image, and obtaining information about a diagnostic state of the object from the ultrasonic image.

Referring to FIGS. 8 and 9, in an embodiment, the processor 120 may control the speed of the cooling fan 50 to a preset lowest speed V₀ during the booting operation (1200).

The booting operation may start at a time point tₐ when a signal for powering on the ultrasonic imaging apparatus 40 is received and end before a time point t_{b1} when an imaging mode selection signal for selecting the imaging mode is received.

The processor 120 may receive a selection of a diagnostic region of the object before the time point t_{b1} when the imaging mode selection signal is received after the booting operation ends.

For example, the processor 120 may receive a selection signal for the diagnostic region of the object generated when the user selects the diagnostic region of the object through the input interface 170.

The diagnostic region of the object may include abdominal organs such as a liver, kidney, spleen, gallbladder, and pancreas, reproductive organs such as a thyroid, breast, prostate, heart, fetus, uterus, and ovary, vessels such as a carotid artery, femoral artery, brachial artery, vertebral artery, and abdominal aorta, musculoskeletal systems such as a muscle, tendon, and ligament, and endocrine organs such as a lymph node and thyroid.

The preset lowest speed V₀ may be the lowest speed among a plurality of preset speeds for the speed of the cooling fan 50. For example, the preset lowest speed V₀ may be a low-noise speed, which is the lowest speed among a preset low-noise speed, medium-noise speed, and high-noise speed.

However, the number of the plurality of preset speeds according to the disclosure is not limited thereto. For example, the number of the plurality of preset speeds may be four or less, or may be four or more.

Hereinafter, for convenience of explanation, the preset lowest speed V₀ will be described as a low-noise speed, a set speed faster than the preset lowest speed V₀ will be described as a medium-noise speed V₀, and a set speed faster than the medium-noise speed V₁ will be described as a high-noise speed V₂.

In an embodiment, the processor 120 may receive a selection of the imaging mode (1300).

For example, the processor 120 may receive a selection signal for the imaging mode generated when the user selects the imaging mode through the input interface 170.

The imaging mode may include at least one of a brightness mode (B-mode), a contrast enhanced ultrasound mode (CEUS), a fusion mode, a motion mode (M-mode), a Doppler mode, an elastography mode, a composite mode, and a 3D mode.

The B-mode may be an imaging mode in which a cross-sectional image of an object is obtained by expressing a reflection intensity of an ultrasonic signal as brightness.

The contrast enhanced ultrasound mode (CEUS) may be a mode in which an image is obtained by observing a movement of a contrast enhanced ultrasound injected into an object. For example, the contrast enhanced ultrasound mode may be an imaging mode in which an image is generated by transmitting an ultrasonic signal to an object into which a contrast enhanced ultrasound is injected and receiving a signal reflected from the contrast enhanced ultrasound.

The fusion mode may be an imaging mode in which an ultrasonic image and another medical image (e.g., CT image, MRI image, etc.) are synthesized to be displayed as one image.

The M-mode may be an imaging mode for observing the movement of an object over time. For example, the M-mode may be an imaging mode in which the movement of an object over time is displayed.

The elastography mode may be a mode in which an elasticity (hardness) of a tissue is measured to be imaged. For example, the elastography mode may be an imaging mode in which a degree of tissue deformation is measured when the tissue is compressed by ultrasound so that a degree of tissue hardness is displayed in color.

The composite mode may be a mode in which two or more imaging modes are displayed simultaneously. For example, the composite mode may be an imaging mode in which the B-mode image and the Doppler mode image are displayed simultaneously, or the B-mode image and the elastography mode image are displayed simultaneously.

The 3D mode may be a mode in which a three-dimensional volume image is generated using two-dimensional images. For example, the 3D mode may be an imaging mode in which continuous two-dimensional images obtained while the probe moves are reconfigured to generate a three-dimensional image.

The Doppler mode may include a color Doppler mode and a power Doppler mode.

The color Doppler mode may be a mode in which a speed and direction of blood flow inside an object are displayed in color using the Doppler effect. For example, the color Doppler mode may be an imaging mode in which blood flow is displayed in red when moving toward the probe and in blue when moving away from the probe.

The power Doppler mode may be a mode in which an amount of blood flow is displayed in color without information about the speed and direction of blood flow using the Doppler effect. For example, the power Doppler mode may be an imaging mode in which slow blood flow or blood flow in a small blood vessel is more sensitively detected and displayed than in the color Doppler mode.

However, the imaging mode according to the disclosure is not limited thereto and may further include various imaging modes. For example, the imaging mode according to the disclosure may further include a pulse inversion harmonic mode, a spatial compound imaging mode, a tissue harmonic imaging mode, a real-time dual mode, a four-dimensional mode, an ECG-gated mode, and the like.

In various embodiments, the processor 120 may control the speed of the cooling fan 50 based on the selected imaging mode when the imaging mode is selected (YES in 1300, and 1400). Details of controlling the speed of the cooling fan 50 based on the selected imaging mode will be described below.

In an embodiment, the processor 120 may increase the speed of the cooling fan 50 based on the selected imaging mode.

For example, after the booting operation, the processor 120 may increase the speed of the cooling fan 50 to the medium-noise speed V₁, which is faster than the low-noise speed V₀, in response to a first imaging mode being selected. A time point when the speed of the cooling fan 50 is increased may coincide with the time point t_{b1} when the first imaging mode selection is received.

This is because while basic operations such as BIOS execution and operating system loading in the booting operation generate a relatively low heating value in the electronic circuit 300, while when the imaging mode is selected, a higher heating value may be generated in the electronic circuit 300 due to operations such as transmission and reception of ultrasonic signals, image processing, and image display.

In an embodiment, the processor 120 may reduce the speed of the cooling fan 50 based on receiving a Freeze signal while operating in the selected imaging mode.

For example, the processor 120 may reduce the speed of the cooling fan 50 to the low-noise speed from a time point t_{f} when receiving the Freeze signal while operating in the first imaging mode.

The Freeze signal may be a signal for keeping the currently displayed ultrasonic image in a frozen state. The freeze signal may be generated in response to a press of the Freeze button 172 (see FIG. 4), a selection of a Freeze icon through the touch screen, an activation of a Freeze function through a voice command, an operation of a foot switch, or a press of the Freeze button provided on the probe 20.

When the Freeze signal is received, the processor 120 may temporarily stop transmitting and receiving ultrasonic signals and freeze the ultrasonic image displayed through the display 140. Accordingly, the amount of computation of the electronic circuit 300 used for transmission and reception of ultrasonic signals and real-time image processing is reduced, so that the heating value is reduced, and therefore the processor 120 may reduce the speed of the cooling fan 50.

In an embodiment, the processor 120 may change the speed of the cooling fan 50 based on the selected imaging mode being switched (YES in 1500, and 1600).

For example, the processor 120 may increase the speed of the cooling fan 50 to the high-noise speed V₂ in response to receiving a signal for switching the imaging mode from the first imaging mode to a second imaging mode. A time point when the speed of the cooling fan 50 is increased to the high-noise speed V₂ may coincide with a time point t_{c1} when a second imaging mode selection is received.

As another example, the processor 120 may reduce the speed of the cooling fan 50 from the high-noise speed V₂ to the low-noise speed V₀ in response to receiving a signal for switching the imaging mode from the second imaging mode to a third imaging mode. A time point when the speed of the cooling fan 50 is reduced to the low-noise speed V₀ may coincide with a time point t_{d1} when a third imaging mode selection is received.

The ultrasonic imaging apparatus 40 according to the disclosure may control the speed of the cooling fan 50 in advance at the time points t_{b1}, t_{c1}, and t_{d1} when the imaging mode selection is received.

Conventionally, the speed of the cooling fan is controlled only at the time points t_{b2}, t_{c2}, and t_{d2} when heat is actually generated and a temperature increases due to image processing after the imaging mode is switched. The image processing may include pre-processing for image obtainment (e.g., beamforming, signal processing, etc.), post-processing of the obtained image (e.g., filtering, image enhancement, etc.), and processing for image display.

For example, conventionally, the speed of the cooling fan is controlled at the time points t_{b2}, t_{c2}, and t_{d2} when a temperature generated due to the image processing after the imaging mode is selected exceeds a reference temperature according to the maximum heating value in the selected imaging mode. Accordingly, cooling begins after heat has already been generated, which reduces cooling efficiency and requires the cooling fan 50 to operate at a high speed to reduce heat generation.

On the other hand, the ultrasonic imaging apparatus 40 according to one embodiment of the present disclosure may reduce noise caused by unnecessary high-speed operation by immediately controlling the speed of the cooling fan 50 at the time point t_{b1}, t_{c1}, and t_{d1} when the imaging mode selection is received.

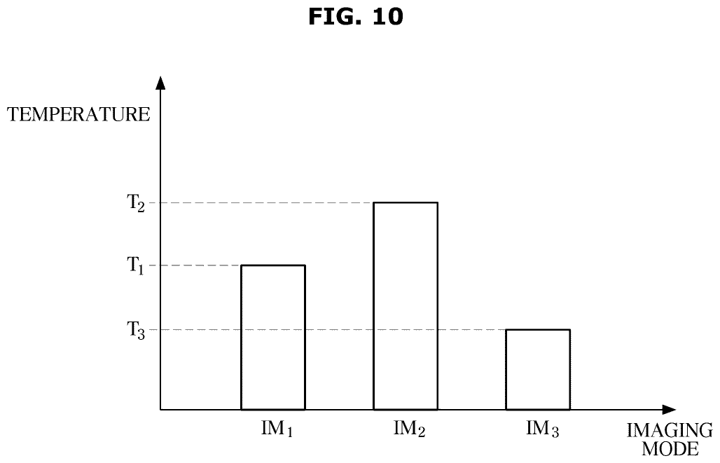

FIG. 10 is a graph for explaining a heating value generated depending on an imaging mode.

Referring to FIG. 10, a temperature according to the maximum heating value generated from the electronic circuit 300 while operating in the imaging mode may vary depending on a type of the imaging mode. One or more temperature sensors for detecting temperatures may be provided inside the housing 41. The temperature sensor may be disposed close to the electronic circuit 300 to detect temperature changes in the electronic circuit 300.

For example, the temperature in the electronic circuit 300 may increase to a first temperature T₁ while operating in a first imaging mode IM₁, may increase to a second temperature T₂ while operating in a second imaging mode IM₂, and may increase to a third temperature T₃ while operating in a third imaging mode IMs. This temperature may be an expected temperature based on the maximum heating value generated from the electronic circuit 300 while operating in each imaging mode.

In an embodiment, the processor 120 may control the speed of the cooling fan 50 based on a pre-stored lookup table and the selected imaging mode.

The lookup table may be a table in which a target rotation speed of the cooling fan 50 for each of a plurality of imaging modes is mapped in proportion to the heating value generated from the electronic circuit 300 while operating in the plurality of imaging modes.

For example, in the lookup table, the high-noise speed V₂, which is the fastest target rotation speed, is mapped to the second imaging mode IM₂ in which the temperature according to the maximum heating value is highest, and the processor 120 may control the speed of the cooling fan 50 to the high-noise speed V₂ at the time point t_{c1} (see FIG. 9) when the second imaging mode IM₂ is selected.

As another example, the medium-noise speed V₁ is mapped to the first imaging mode IM₁ in which the temperature according to the maximum heating value is lower than the temperature in the second imaging mode IM₂, and thus the processor 120 may control the speed of the cooling fan 50 to the medium-noise speed V₁ at the time point t_{b1} (see FIG. 9) when the first imaging mode IM₁ is selected.

As another example, the low-noise speed V₀, which is the lowest target rotation speed, is mapped to the third imaging mode IM₃ in which the temperature according to the maximum heating value is lowest, and the processor 120 may control the speed of the cooling fan 50 to the low-noise speed V₀ at the time point t_{d1} (see FIG. 9) when the third imaging mode IM₃ is selected.

As such, by using the lookup table in which the target rotation speed for each of the plurality of imaging modes is mapped in proportion to the temperature according to the maximum heating value generated from the electronic circuit 300 while operating in the plurality of imaging modes, the processor 120 may determine and control an appropriate speed of the cooling fan 50 immediately at the time point when the imaging mode is selected.

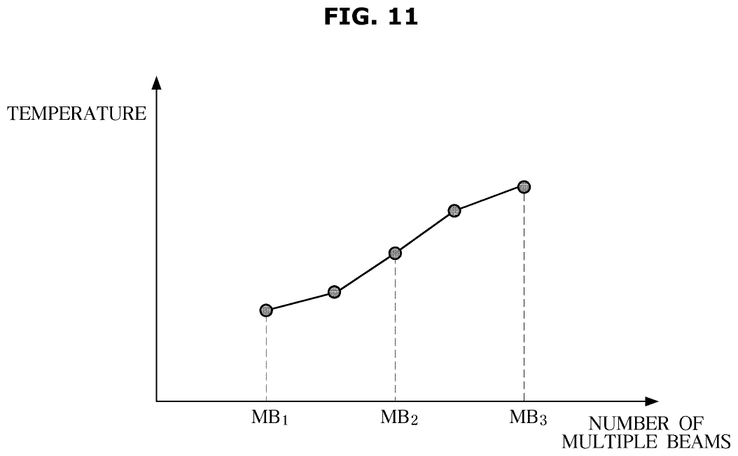

FIG. 11 is a graph for explaining the heating value generated depending on a diagnostic region of an object in a selected imaging mode.

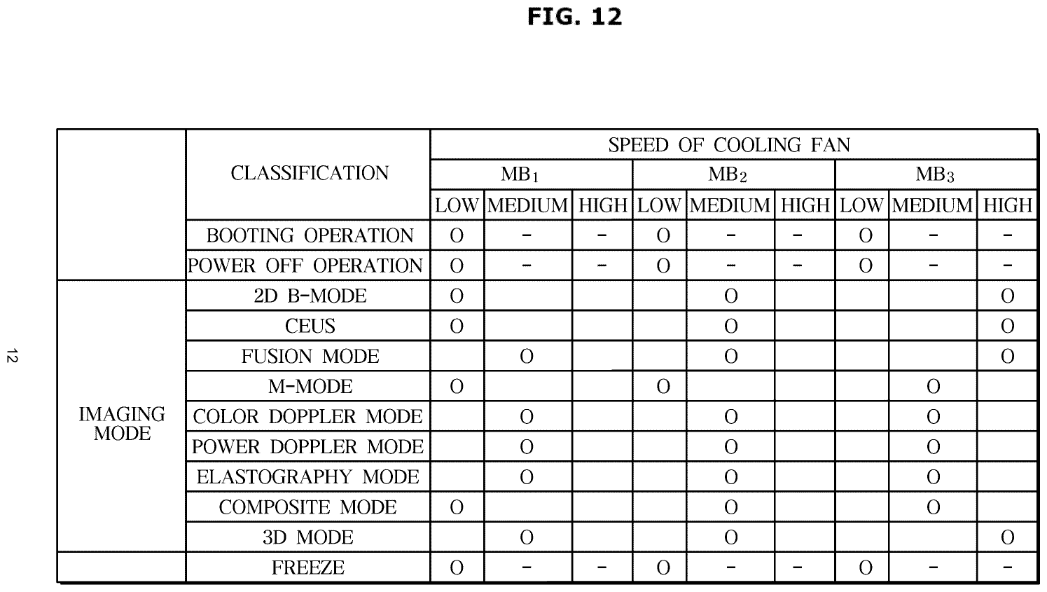

FIG. 12 illustrates a lookup table used for speed control of the cooling fan.

In an embodiment, the processor 120 may receive the selection of a diagnostic region of the object.

The receiving of the selection of the diagnostic region of the object may include receiving the selection signal for the diagnostic region of the object generated when the user selects the diagnostic region of the object through the input interface 170.

The ultrasonic imaging apparatus 40 according to an embodiment may obtain an ultrasonic image through a multi-beam method.

The multi-beam method is a method of obtaining an ultrasonic image by transmitting and transmitting multiple beams, which are a plurality of beams, rather than a single beam when scanning the object. When an ultrasonic image is obtained through the multi-beam method, image quality of the ultrasonic image may be improved.

When an ultrasonic image is obtained through the multi-beam method, the number of multiple beams required to obtain the ultrasonic image by each diagnostic region of the object may vary.

For example, the larger a depth and size of the diagnostic region of the object, the greater the number of multiple beams required to obtain an ultrasonic image is ultrasonic image.

A method of controlling the speed of the cooling fan 50 based on the selected imaging mode and the selected diagnostic region of the object will be described below.

Referring to FIG. 11, the processor 120 may obtain an ultrasonic image depending on the number of multiple beams required to obtain the ultrasonic image based on the selected diagnostic region of the object.

For example, the processor 120 may, in a case in which the selected diagnostic region of the object is a first region, determine a first number MB₁ required to obtain an ultrasonic image, and transmit and receive multiple beams of the determined first number MB₁ to obtain the ultrasonic image, and may, in a case in which the selected diagnostic region of the object is a second region, determine a third number MB₃ required to obtain an ultrasonic image, which is greater than the first number MB₁, and transmit and receive multiple beams of the determined third number MB₃ to obtain the ultrasonic image. Also, the processor 120 may, in a case in which the selected diagnostic region of the object is a third region, determine the number of multiple beams required to obtain an ultrasonic image as a second number MB₂, which is greater than the first number MB₁ and less than the third number MB₃, and transmit and receive multiple beams of the determined second number MB₂ to obtain the ultrasonic image.

When an ultrasonic image is obtained, as the number of multiple beams increases, the amount of computation of the electronic circuit 300 increases, which may increase the heating value.

For example, the temperature according to the heating value of the electronic circuit 300 may be higher in the case of obtaining an ultrasonic image by transmitting and receiving the multiple beams of the second number MB₂ than in the case of obtaining an ultrasonic image by transmitting and receiving the multiple beams of the first number MB₁.

Referring to FIG. 12, in an embodiment, the processor 120 may control the speed of the cooling fan 50 based on the selected imaging mode and the selected diagnostic region of the object.

For example, in a case in which the selected imaging mode is the fusion mode and the diagnostic region of the object is the first region, the processor 120 may determine the number of multiple beams for obtaining an ultrasonic image in the diagnostic region of the object as the first number MB and control the speed of the cooling fan 50 to the medium-noise speed V₁.

As another example, in a case in which the selected imaging mode is the fusion mode and the diagnostic region of the object is the third region, the processor 120 may determine the number of multiple beams for obtaining an ultrasonic image in the diagnostic region of the object as the second number MB₂ and control the speed of the cooling fan 50 to the medium-noise speed V₁.

In an embodiment, the processor 120 may control the speeds of the cooling fan 50 differently when the selected diagnostic regions of the object are different in the selected imaging mode.

For example, in a case in which the selected imaging mode is the B-mode and the diagnostic region of the object is the first region, the processor 120 may determine the number of multiple beams for obtaining an ultrasonic image in the diagnostic region of the object as the first number MB₁ and control the speed of the cooling fan 50 to the low-noise speed V₀.

On the other hand, in a case in which the selected imaging mode is the B-mode and the diagnostic region of the object is the third region, the processor 120 may determine the number of multiple beams for obtaining an ultrasonic image in the diagnostic region of the object as the second number MB₂ and control the speed of the cooling fan 50 to the medium-noise speed V₁. Also, in a case in which the selected imaging mode is the B-mode and the diagnostic region of the object is the second region, the processor 120 may determine the number of multiple beams for obtaining an ultrasonic image in the diagnostic region of the object as the third number MB₃ and control the speed of the cooling fan 50 to the high-noise speed V₂.

When the selected imaging mode is the contrast enhanced ultrasound mode, the processor 120 may control the speed of the cooling fan 50 to one of the low-noise speed V₀, the medium-noise speed V₁, and the high-noise speed V₂ depending on the selected diagnostic region of the object.

When the selected imaging mode is the M-mode, the processor 120 may control the speed of the cooling fan 50 to one of the low-noise speed V₀ and the medium-noise speed V₁ depending on the selected diagnostic region of the object.

When the selected imaging mode is the composite mode, the processor 120 may control the speed of the cooling fan 50 to one of the low-noise speed V₀ and the medium-noise speed V₁ depending on the selected diagnostic region of the object.

When the selected imaging mode is the 3D mode, the processor 120 may control the speed of the cooling fan 50 to one of the medium-noise speed V₁ and the high-noise speed V₂ depending on the selected diagnostic region of the object.

In an embodiment, the processor 120 may control the speed of the cooling fan 50 to a target speed corresponding to the selected imaging mode regardless of the selected diagnostic region depending on the selected imaging mode.

For example, when the selected imaging mode is the Doppler mode (the color Doppler mode and power Doppler mode), the processor 120 may control the speed of the cooling fan 50 to the medium-noise speed V₁, which is the target speed, regardless of the selected diagnostic region of the object.

As another example, when the selected imaging mode is the elastography mode, the processor 120 may control the speed of the cooling fan 50 to the medium-noise speed V₁, which is the target speed, regardless of the selected diagnostic region of the object.

In an embodiment, when the selected imaging mode is the B-mode or the Doppler mode, the processor 120 may control the speed of the cooling fan 50 to a faster speed in the Doppler mode than in the B-mode or to a faster speed in the B-mode than in the Doppler mode, depending on the selected diagnostic region of the object.

For example, as described above, in the case in which the selected diagnostic region of the object is the first region, the processor 120 may control the speed of the cooling fan 50 to the low-noise speed V₀ when the selected imaging mode is the B-mode, and may control the speed of the cooling fan 50 to the medium-noise speed V₁ when the selected imaging mode is the Doppler mode (the color Doppler mode or power Doppler mode). Also, as described above, in the case in which the selected diagnostic region of the object is the second region, the processor 120 may control the speed of the cooling fan 50 to the high-noise speed V₂ when the selected imaging mode is the B-mode, and may control the speed of the cooling fan 50 to the medium-noise speed V₁ when the selected imaging mode is the Doppler mode (the color Doppler mode or power Doppler mode).

However, the controlling of the speed of the cooling fan 50 based on the selected imaging mode and the selected diagnostic region of the object according to the disclosure is not limited to that illustrated in FIG. 12, and may include controlling the speed of the cooling fan 50 based on selected imaging modes and selected diagnostic regions of the object, according to various embodiments.

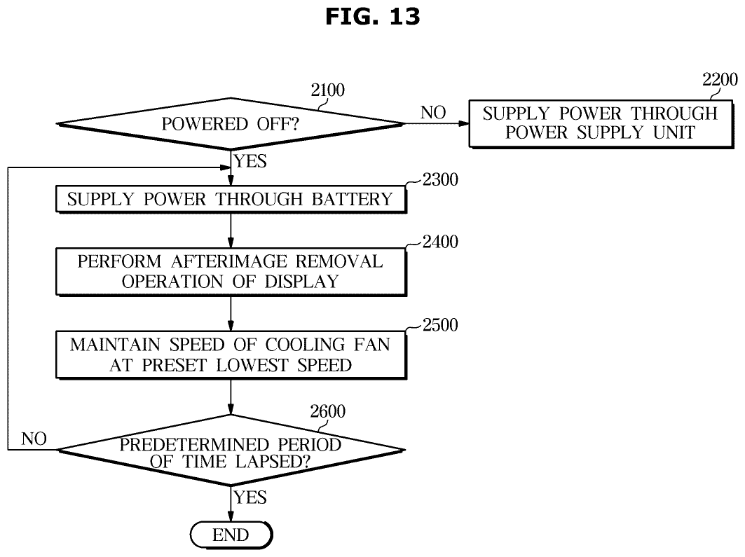

FIG. 13 is a flowchart illustrating an example of a control method of the ultrasonic imaging apparatus according to an embodiment.

Referring to FIG. 13, in an embodiment, the processor 120 may supply power through the battery 250 based on the ultrasonic imaging apparatus 40 being powered off (YES in 2100, and 2300). The battery 250 may be an auxiliary power device capable of supplying power independently from the power supply unit 200.

The processor 120 may supply power to the electronic circuit 300 and the cooling fan 50 through the power supply unit 200 before the ultrasonic imaging apparatus 40 is powered off (NO in 2100, and 2200).

The processor 120 may perform the afterimage removal operation of the display 140 while receiving power through the battery 250 (2400).

The afterimage removal operation may be an operation for removing afterimages caused by the same image being displayed on the display 140 for a long period of time. For example, the processor 120 may perform the afterimage removal operation for a predetermined period of time by a method of sequentially displaying the RGB patterns on all the pixels of display 140, displaying the entire screen in white and then in black, causing all the pixels to emit light at maximum brightness, or repeatedly displaying an image of a specific pattern.

In an embodiment, the processor 120 may maintain the speed of the cooling fan 50 at the preset lowest speed for a predetermined period of time in response to the ultrasonic imaging apparatus 40 being powered off (2500 and 2600).

For example, the processor 120 may maintain the speed of the cooling fan 50 at the preset lowest speed while the ultrasonic imaging apparatus 40 is powered off to perform the afterimage removal operation on the display 140. The preset lowest speed may be the low-noise speed V₀.

This is because the amount of computation of the electronic circuit 300 is small during the afterimage removal operation and thus the heating value is not large, so that sufficient cooling is possible even when the cooling fan 50 is operated at the lowest speed.

The predetermined period of time may be set to be sufficient time for the afterimage removal operation to be completed. For example, the predetermined period of time may be set from several seconds to several minutes, and may be set differently depending on characteristics of the display 140 or a degree of afterimage.

As such, the processor 120 may prevent unnecessary noise from being generated during the afterimage removal operation of the display 140 by maintaining the speed of the cooling fan 50 at the preset lowest speed while performing the afterimage removal operation of the display 140. Additionally, the processor 120 may minimize power consumption of the battery 250 by stopping supplying power through the battery 250 after the afterimage removal operation is completed.

As is apparent from the above, according to an aspect of the disclosure, there is an effect of reducing noise caused by rotation of a cooling fan by controlling a speed of the cooling fan depending on a selected imaging mode.

According to an aspect of the disclosure, there is the effect of reducing noise caused by rotation of the cooling fan by preemptively controlling the speed of the cooling fan before heat is generated from various components of an ultrasonic imaging apparatus.

Effects capable of being achieved in this disclosure are not limited to the effects mentioned above, and other effects not mentioned will be clearly understood by those skilled in the art from the description above.

The disclosed embodiments may be implemented in the form of a recording medium storing instructions executable by a computer. The instructions may be stored in the form of program code, and when executed by a processor, a program module may be created to perform the operations of the disclosed embodiments. The recording medium may be implemented as a computer-readable recording medium.

The computer-readable recording medium includes any type of recording medium in which instructions readable by the computer are stored. For example, the recording medium may include a read only memory (ROM), a random access memory (RAM), a magnetic tape, a magnetic disk, a flash memory, an optical data storage device, and the like.

The embodiments disclosed with reference to the accompanying drawings have been described above. It will be understood by those skilled in the art that various changes in form and details may be made therein without departing from the spirit and scope of the disclosure as defined by the appended claims. The disclosed embodiments are illustrative and should not be construed as limiting.

What is claimed is:

1. An ultrasonic imaging apparatus comprising:
an electronic circuit configured to control an operation of the ultrasonic imaging apparatus;
a housing configured to accommodate the electronic circuit;
a cooling fan disposed inside the housing to cool the electronic circuit; and
a processor configured to receive a selection of an imaging mode of the ultrasonic imaging apparatus and control a speed of the cooling fan based on the selected imaging mode.

2. The ultrasonic imaging apparatus according to claim 1, wherein
the processor is configured to change the speed of the cooling fan from a first speed to a second speed in response to the selected imaging mode being switched from a first imaging mode to a second imaging mode.

3. The ultrasonic imaging apparatus according to claim 1, wherein
the processor is configured to control the speed of the cooling fan based on a pre-stored lookup table and the selected imaging mode, and
the pre-stored lookup table is a table in which a target rotation speed for each of a plurality of imaging modes is mapped in proportion to a heating value generated from the electronic circuit while operating in the plurality of imaging modes.

4. The ultrasonic imaging apparatus according to claim 1, wherein
the processor is configured to reduce the speed of the cooling fan based on receiving a Freeze signal while operating in the selected imaging mode.

5. The ultrasonic imaging apparatus according to claim 1, wherein
the processor is configured to receive a selection of a diagnostic region of an object and control the speed of the cooling fan based on the selected imaging mode and the selected diagnostic region of the object.

6. The ultrasonic imaging apparatus according to claim 5, wherein
the processor is configured to
control the speed of the cooling fan to a first speed based on the selected imaging mode being a first imaging mode and the selected diagnostic region of the object being a first region, and
control the speed of the cooling fan to a second speed based on the selected imaging mode being the first imaging mode and the selected diagnostic region of the object being a second region different from the first region.

7. The ultrasonic imaging apparatus according to claim 5, wherein
the processor is configured to
control the speed of the cooling fan to a first speed based on the selected imaging mode being a B-mode and the selected diagnostic region of the object being a first region,
control the speed of the cooling fan to a second speed based on the selected imaging mode being the B-mode and the selected diagnostic region of the object being a second region, and
control the speed of the cooling fan to a third speed based on the selected imaging mode being a Doppler mode and the selected diagnostic region of the object being the first region or the second region, and
the third speed is faster than the first speed and slower than the second speed.

8. The ultrasonic imaging apparatus according to claim 5, wherein
the processor is configured to control the speed of the cooling fan to a target speed corresponding to a Doppler mode regardless of the selected diagnostic region when the selected imaging mode is the Doppler mode.

9. The ultrasonic imaging apparatus according to claim 1, wherein
the processor is configured to control the speed of the cooling fan to a preset lowest speed during a booting operation of booting a program necessary to execute the imaging mode.

10. The ultrasonic imaging apparatus according to claim 1, wherein
the processor is configured to maintain the speed of the cooling fan at a preset lowest speed for a predetermined period of time in response to the ultrasonic imaging apparatus being powered off.

11. A control method of an ultrasonic imaging apparatus which comprises: an electronic circuit configured to control an operation of the ultrasonic imaging apparatus; a housing configured to accommodate the electronic circuit; and a cooling fan disposed inside the housing to cool the electronic circuit,
wherein the control method comprises:
receiving a selection of an imaging mode of the ultrasonic imaging apparatus; and
controlling a speed of the cooling fan based on the selected imaging mode.

12. The control method according to claim 11, further comprising
changing the speed of the cooling fan from a first speed to a second speed in response to the selected imaging mode being switched from a first imaging mode to a second imaging mode.

13. The control method according to claim 11, wherein
the controlling of the speed of the cooling fan based on the selected imaging mode comprises controlling the speed of the cooling fan based on a pre-stored lookup table and the selected imaging mode, and
the pre-stored lookup table is a table in which a target rotation speed for each of a plurality of imaging modes is mapped in proportion to a heating value generated from the electronic circuit while operating in the plurality of imaging modes.

14. The control method according to claim 11, further comprising
reducing the speed of the cooling fan based on receiving a Freeze signal while operating in the selected imaging mode.

15. The control method according to claim 11, wherein
the controlling of the speed of the cooling fan based on the selected imaging mode comprises:
receiving a selection of a diagnostic region of an object; and
controlling the speed of the cooling fan based on the selected imaging mode and the selected diagnostic region of the object.

16. The control method according to claim 15, wherein
the controlling of the speed of the cooling fan based on the selected imaging mode and the selected diagnostic region of the object comprises:
controlling the speed of the cooling fan to a first speed based on the selected imaging mode being a first imaging mode and the selected diagnostic region of the object being a first region; and
controlling the speed of the cooling fan to a second speed based on the selected imaging mode being the first imaging mode and the selected diagnostic region of the object being a second region different from the first region.

17. The control method according to claim 15, wherein
the controlling of the speed of the cooling fan based on the selected imaging mode and the selected diagnostic region of the object comprises:
controlling the speed of the cooling fan to a first speed based on the selected imaging mode being a B-mode and the selected diagnostic region of the object being a first region;
controlling the speed of the cooling fan to a second speed based on the selected imaging mode being the B-mode and the selected diagnostic region of the object being a second region; and
controlling the speed of the cooling fan to a third speed based on the selected imaging mode being a Doppler mode and the selected diagnostic region of the object being the first region or the second region, and
the third speed is faster than the first speed and slower than the second speed.

18. The control method according to claim 15, wherein
the controlling of the speed of the cooling fan based on the selected imaging mode and the selected diagnostic region of the object comprises
controlling the speed of the cooling fan to a target speed corresponding to a Doppler mode regardless of the selected diagnostic region when the selected imaging mode is the Doppler mode.

19. The control method according to claim 11, further comprising
controlling the speed of the cooling fan to a preset lowest speed during a booting operation of booting a program necessary to execute the imaging mode.

20. The control method according to claim 11, further comprising
maintaining the speed of the cooling fan at a preset lowest speed for a predetermined period of time in response to the ultrasonic imaging apparatus being powered off.
